(19) United States
(12) Patent Application Publication
(10) Pub. No.: US 2026/0092098 A1
(43) Pub. Date: Apr. 2, 2026

(54) HUMAN A INTERFERON RECEPTOR BINDING-RELATED SITE MUTANT AND USE THEREOF

(71) Applicant: FUDAN UNIVERSITY, Shanghai (CN)

(72) Inventors: Zhenghong YUAN, Shanghai (CN); Jieliang CHEN, Shanghai (CN); Yaming LI, Shanghai (CN)

(21) Appl. No.: 19/133,302

(22) PCT Filed: Jan. 22, 2020
(86) PCT No.: PCT/CN2020/073903

§ 371 Date: Dec. 10, 2025

(30) Foreign Application Priority Data
Jan. 30, 2019 (CN) 201910090516.2

(51) Int. Cl. C07K 14/715; A61K 38/00; A61P 31/20

(52) U.S. Cl. CPC C07K 14/7156; A61P 31/20; A61K 38/00

(57) ABSTRACT

Provided is a human a interferon receptor binding-related site mutant IFN-α2-EIFK. The mutant has stronger anti-hepatitis B virus activity than IFN-α2, and has no cytotoxic effect at an antiviral concentration. An anti-hepatitis B virus drug can be prepared therefrom.

### TECHNICAL FIELD

The present invention belongs to the technical field of medicine and bioengineering, and relates to a human interferon alpha receptor-binding site mutant and uses thereof. In particular, it relates to an interferon alpha mutant prepared by modifying and purifying human interferon alpha mutant, and the use in the preparation of antiviral agents against hepatitis B virus. Such medicament reduces or eliminates viral surface antigen (HBsAg) and DNA in hepatocytes infected with the hepatitis B virus.

### BACKGROUND OF THE INVENTION

Hepatitis B virus (HBV) is a major pathogen that seriously endangers human health. According to relevant statistics, there are approximately 240 million HBV carriers worldwide, among which nearly 80 million chronically HBV-infected individuals in China. Although hepatitis B vaccine can prevent HBV infection, there are still hundreds of thousands of new cases of chronic hepatitis B infection annually; meanwhile, hundreds of thousands of people die each year from liver diseases caused by chronic hepatitis B. Due to the lack of specific therapy, how to achieve functional cure for chronic hepatitis B, i.e., seroclearance of hepatitis B surface antigen (HBsAg) and persistent silencing of cccDNA, and even complete cure, i.e., the clearance of the viral genomic DNA (covalently closed circular DNA, cccDNA), remains a challenge in this technical field. Wherein, for example, many patients require long-term or lifelong administration of nucleoside/tide analog antiviral drugs to control the viral infection and treat the disease, resulting in serious economic burden and reduced quality of life.

Interferons (IFNs) are cytokines with direct antiviral effects and immunomodulatory effects, first discovered and named in 1957. They play a key role in the host antiviral immune response. Currently, more than ten types of IFNs have been identified. IFNs are broadly classified into Type I and Type II based on their receptors: Type I interferons mainly include IFN-α and IFN-β (IFN-λ is generally classified as a Type III interferon), while Type II interferon is mainly consists of IFN-γ. With the advent of genetic engineering technology, recombinant human IFN-α was cloned and applied in the treatment of diseases such as viral hepatitis in the last century. Compared with another major class of drugs for treating chronic hepatitis B—nucleoside (tide) analogs, IFN-α and its PEGylated form (PEG-IFN-α) not only have direct antiviral effects but also possess immunomodulatory functions, thus having the advantage of relatively higher rates of HBsAg seroclearance and sustained virological response. However, clinical statistics show that IFN-α therapy for chronic hepatitis B remains suboptimal. For example, after 48 months of treatment with pegylated interferon, only about 30% of HBeAg-positive patients achieve HBeAg seroclearance, while the HBsAg seroclearance rate is below 5%. Therefore, it is a consensus in the field that there is an urgent need to optimize and improve the antiviral efficacy and response rate of interferon therapy.

Studies have shown that interferon exerts its antiviral effect by specifically binding to IFNAR on the cell surface, thereby initiating the transduction of the downstream JAK-STAT signaling pathway and inducing the transcriptional expression of Interferon-Stimulated Genes (ISGs). Currently, thirteen human IFN-α subtypes, including IFN-α2, have been successively identified. The encoding genes for these subtypes are all located on human chromosome 9. While the various interferon-α subtypes share many similar structural domains, approximately 30% of their amino acid sequences are non-conserved. Reports indicate that although all IFN-α subtypes exert their functions by binding to both subunits of the type I interferon receptor, IFNAR1 and IFNAR2, the extent and manner of downstream canonical or non-canonical signaling pathways activation vary among subtypes, reflecting differences in their binding affinities for the two receptor subunits. Meanwhile, the sensitivity to IFN subtypes also varies among different viruses and different cell types. Studies on the affinity of interferons have shown that interferon generally binds with high binding affinity to IFNAR2 but exhibits relatively low affinity for IFNAR1, and the amino acid residues within the interferon that mediate receptor binding have been substantially elucidated.

Previous studies have shown that HBV exhibits varying sensitivities to different IFN-α subtypes, with IFN-α14 demonstrating the most potent inhibiting of HBV replication at the same effective concentration. Additional studies have reported that the thirteen IFN-α subtypes possess different binding dissociation constants for the two subunits of the interferon receptor. Regression analyses correlating inhibition of secreted viral antigens and intracellular HBV RNA levels by each IFN-α subtype revealed that the anti-HBV efficacy of IFN-α subtypes is positively correlated with their affinity for IFNAR1, but not for IFNAR2. Further, comparative analysis of the amino acid sequences of IFN-α2 and IFN-α14 identified four amino acid differences at IFNAR1-binding site. Substitution of these four residues in IFN-α2 with the corresponding amino acids from IFN-α14 yielded an IFN-α2 mutant. Subsequently, testing the antiviral function and signaling pathway activation of this IFN-α2 mutant revealed that it possessed antiviral effects and signaling pathway activation effects similar to those of IFN-α14. These research findings expand the scientific understanding of the antiviral mechanism of interferon-α, and provide a theoretical and technical basis for development of novel therapeutic strategies for chronic hepatitis B based on interferon mutants altered receptor-binding properties.

Based on the current state and foundation of the prior art, the inventors of the present application intend to provide a human interferon alpha receptor-binding site mutant and uses thereof, wherein the mutation is obtained through modification and purification of interferon alpha, and is useful in the preparation of a medicament against hepatitis B virus infection.

### DISCLOSURE OF THE INVENTION

The purpose of the present invention is, based on the current state and foundation of the prior art, to provide a human interferon alpha receptor-binding site mutant.

Another purpose of the present invention is to provide the use of the human interferon alpha receptor-binding site mutant. The mutant is designed to enhance direct anti-HBV effect by mutating specific amino acid residues involved in the interaction between human IFN-α2 and the interferon receptor 1 subunit.

The results of the present invention shows that the interferon mutant obtained through targeted specific individual amino acid residues exhibits stronger anti-HBV activity and requires a lower working concentration than the IFN-α2 currently used in clinical practice, and is capable of reducing or eliminating viral surface antigen (HBsAg) level and HBV DNA level in hepatocytes infected with hepatitis B virus.

The present invention thereby provides new approaches and theoretical and technical support for the development of novel medicaments for the treatment of chronic hepatitis B based on specific interferon alpha mutants.

The present invention is based on the following research foundations. First, preliminary studies for this application have showed that HBV displays different sensitivities to various IFN-αsubtypes, wherein IFN-α14 exhibits the most significant inhibitory effect on HBV replication at the same effective concentration. Second, it has been reported that the thirteen IFN-α subtypes possess distinct binding dissociation constants with the two interferon receptor subunits, and a regression analysis revealed that the anti-HBV effect of the subtypes is positively correlated with their affinity for IFNAR1, but not for IFNAR2. Third, comparative analysis of the amino acid sequences of IFN-α2 and IFN-α14 at IFNAR1-binding identified four amino acid residues; when these four residues in IFN-α2 were mutated to the corresponding amino acids of IFN-α14, the IFN-α2 mutant was found to exhibit antiviral effects and signaling pathway activation similar to those of IFN-α14. Building upon these findings, the present invention constructs prokaryotic expression plasmids encoding human IFN-α and its mutant, and produces biologically active human IFN-α subtypes and the corresponding mutant recombinant proteins using methods of prokaryotic expression and purification methods.

Specifically, in the present invention, based on observed differences in the anti-HBV activity among various IFN-α subtypes and the positive correlation of this activity with IFNAR1-binding affinity, the amino acid sequence of IFN-α14, which exhibits a stronger anti-HBV efficacy, and clinically used IFN-α2 were compared. This comparison revealed four amino acids differences at IFNAR1-binding sites between the two IFN-α subtypes. Accordingly, the present invention mutates substitutes Aspartic acid at position 82 of human IFN-α2 to Glutamic acid, Threonine at position 86 to Isoleucine, Tyrosine at position 89 to Phenylalanine, and Arginine at position 120 to Lysine, thereby obtaining an interferon mutant, IFN-α2-EIFK, with enhanced affinity for IFNAR1. Subsequently, the anti-HBV efficacy of human IFN-α2 and IFN-α2-EIFK was evaluated in HBV-infected cell models, including HepG2-NTCP cells and primary human hepatocytes (PHH), by assessing the inhibitory effects on HBeAg, HBsAg, and HBV DNA. The results show that the interferon mutant, IFN-α2-EIFK, possesses a potent anti-HBV effect similar to IFN-α14, and its inhibitory effects on viral HBs and HBe antigens and viral DNA are 2-10 fold greater than those of IFN-α2, and it exhibits no cytotoxicity at working concentrations.

In the present invention, the amino acid sequence of the human IFN-α2 recombinant protein used is derived from the human genome and is set forth in SEQ ID NO: 1.

In the present invention, the amino acid sequence of IFN-α14 used for comparison with IFN-α2 is set forth in SEQ ID NO: 3.

In the present invention, the amino acid sequence of IFN-α2-EIFK, which is mutated at four IFNAR1 receptor binding-related amino acid sites in IFN-α2, is set forth in SEQ ID NO: 2.

In the present invention, human IFN-α2 and IFN-α2-EIFK are prepared and purified by the following method:

The encoding sequence of human IFN-α2 (SEQ ID NO: 1), IFN-α2-EIFK (SEQ ID NO: 2) (as shown in FIG. 1A), and IFN-α14 (SEQ ID NO: 3) were cloned into a prokaryotic expression vector. Interferons are harvested after prokaryotic expression of the recombinant protein, followed by protein concentration and removal of endotoxin. The purified interferon is then diluted to various concentrations, aliquoted, and stored at −80° C.

The present invention includes the purification of interferon from a prokaryotic expression system and assessment of its purity. Comparative experiments were conducted to evaluate the inhibition of HBV antigens and DNA levels by the two interferons, human IFN-α2 and IFN-α2-EIFK, in an HBV infection and replication model. Additionally, the activation effects of the two interferons, human IFN-α2 and IFN-α2-EIFK, on the classical JAK-STAT1/STAT2 pathway were compared, as well as the induction of specific Interferon-Stimulated Genes (ISGs).

In HepG2-NTCP cells, experimental data from the present invention obtained from Western blotting, an Interferon-Stimulated Response Element (ISRE) fluorescent reporter system, and quantitative PCR show that, compared to human IFN-α2, the interferon mutant IFN-α2-EIFK exhibits stronger activation of the classical interferon JAK-STAT pathway, greater induction on ISRE, and higher levels of ISGs. Specifically, IFN-α2-EIFK induced a higher level of ISGs associated with anti-HBV therapeutic efficacy compared to the prior art.

The results from experiments and testing show that the human interferon alpha receptor-binding site mutant of the present invention, the interferon mutant IFN-α2-EIFK, exhibits superior activity to currently used IFN-α2 in inhibiting the levels of HBV surface antigen, e-antigen, and viral DNA, without cytotoxic effects. To achieve a similar antiviral effect, the working concentration of IFN-α2-EIFK is more than 10-fold lower than that of IFN-α2. Furthermore, the IFN-α mutant can be used for the preparation of a novel pharmaceutical compositions for the treatment of chronic hepatitis B.

To facilitate understanding, the superior anti-HBV activity of the IFN-α2-EIFK mutant of the present invention compared to IFN-α2 will be described in detail below with reference to specific figures. It should be noted that these figures are for illustrative purposes only, and it is evident that professionals and technicians in this field could make modification to individual sites, processes, etc., within the scope of the present invention, and such modifications also considered within the scope of the present invention.

### BRIEF DESCRIPTION OF THE DRAWINGS

FIG. 1. Purification of interferon from a prokaryotic expression system and assessment of its purity;
wherein, FIG. 1A is a schematic diagram illustrating the sequence alignment of human IFN-α2, IFN-α14, and the IFN-α2-EIFK mutant; and FIG. 1B shows the Coomassie Brilliant Blue staining results for purified human IFN-α2 and IFN-α2-EIFK.

FIG. 2. Comparison of the inhibition of HBV antigen and DNA levels by the two interferons, human IFN-α2 and IFN-α2-EIFK, in an HBV infection and replication model; wherein, FIG. 2A illustrates the antiviral effects of IFN-α2 and IFN-α2-EIFK in HepG2-NTCP cells; and FIG. 2B illustrates the antiviral effects of IFN-α2 and IFN-α2-EIFK in PHH cells.

FIG. 3. Comparison of the activation effects of the two interferons, human IFN-α2 and IFN-α2-EIFK, on the classical JAK-STAT1/STAT2 pathway; wherein, FIG. 3A illustrates the differences in the stimulation of STAT1 and STAT2 phosphorylation levels by IFN-α2 and IFN-α2-EIFK; and FIG. 3B illustrates the differences in ISRE activation induced by IFN-α2 and IFN-α2-EIFK.

FIG. 4. Comparison of the differences in the induction of certain interferon-stimulated genes (ISGs) by the two interferons, human IFN-α2 and IFN-α2-EIFK.

### DETAILED DESCRIPTION

### Example 1: Preparation and Purification of Human IFN-α2 and IFN-α2-EIFK

The gene encoding human IFN-α2 (SEQ ID NO: 1), the interferon mutant IFN-α2-EIFK (SEQ ID NO: 2) (as shown in FIG. 1A), and the IFN-α14 (SEQ ID NO: 3) were cloned into a prokaryotic expression vector, followed by recombinant protein expression method in prokaryotic system.
(1) The recombinant plasmid was transformed into E. coli BL-21 and plated on solid LB medium containing antibiotic. After incubation at 37° C. for 16 hours, single colonies were picked and inoculated into 3-4 ml of LB medium for small-scale shaking culture overnight. Then, the previous culture was inoculated into a large-scale shaking culture at a 1:100 ratio. When the OD600 reached between 0.5 and 0.6, induction was performed with IPTG at a final concentration of 10 μM. Incubation was performed at 16° C. for 20 hours. After completion of protein expression, the bacterial culture was transferred to a 50 ml centrifuge tube, centrifuged at 5000 g at 4° C. for 10 minutes, and the supernatant was discarded.
(2) The bacterial pellet was resuspended in Buffer A (20 mM phosphate buffer, 0.5 M sodium chloride, 20 mM imidazole); the volume of the resuspension was 1/50- 1/100 of the bacterial culture volume. The cell pellet was resuspended and thoroughly dispersed by pipetting, then transferred to a 2 ml centrifuge tube. The cells were then sonicated on ice at high-power setting, sonication for 10 s, cooling for 10 s, repeated for 6 cycles; this procedure was repeated 3 times. The lysate was then centrifuged at 10,000 g for 25 minutes at 4° C., and the supernatant was collected. The collected supernatant was diluted with Buffer A to 1/20 of the original culture volume, filtered first through a 0.45 μm filter membrane and then through a 0.22 μm filter membrane, and stored at 4° C. for later use.
(3) Affinity purification was performed using the AKTA avant system (GE company) with a HistrapHP column (catalog number: 17524701; GE company). Subsequently, ion exchange chromatography was performed using the AKTA Avant system (GE company) with a GE Hitrap Q HP column (catalog number: 17115401, GE company).
(4) The collected samples were subjected to Coomassie Brilliant Blue staining to assess interferon purity, as shown in FIG. 1B.
(5) The purified interferon was concentrated using an ultrafiltration device, and the buffer was exchanged to PBS.
(6) To eliminate the effect of endotoxin on experimental results, endotoxin was subsequently removed from the interferon by using Triton X-114. Interferon was mixed with 10% Triton X-114 at a ratio of 9:1 and stirred magnetically at 4° C. for 60 minutes to mix thoroughly. The mixture was incubated in a 30° C. metal bath/heating block and shaken at 1000 rpm for 20 minutes. Afterward, it was then taken out, mixed by inversion, and again incubated in a 30° C. metal bath/heating block and shaken at 1000 rpm for 20 minutes. The mixture was centrifuged at 14,000 g at 25° C. for 15 minutes, and the upper aqueous phase was carefully transferred to a new tube. Then, these steps were repeated once more.
(7) The aforementioned interferon was transferred into a pre-treated dialysis bag, with pre-cooled PBS as the dialysis buffer outside the bag, and dialyzed overnight.
(8) Following purification by dialysis, the interferon was diluted to various concentrations, and the protein concentration thereof was determined by BCA assay. After all steps were completed, the interferon was aliquoted and stored in a −80° C. freezer.

### Example 2: HBV Infection System

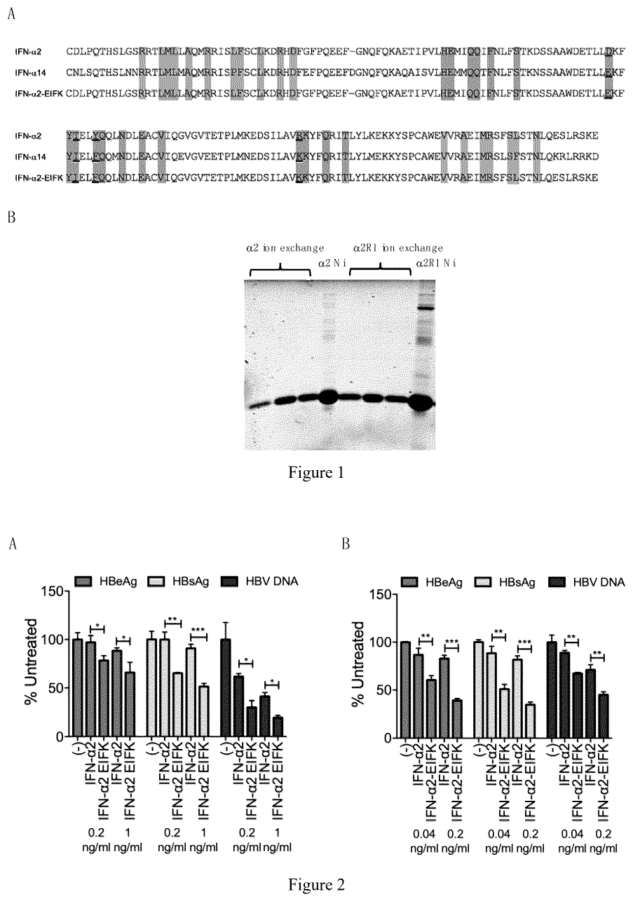

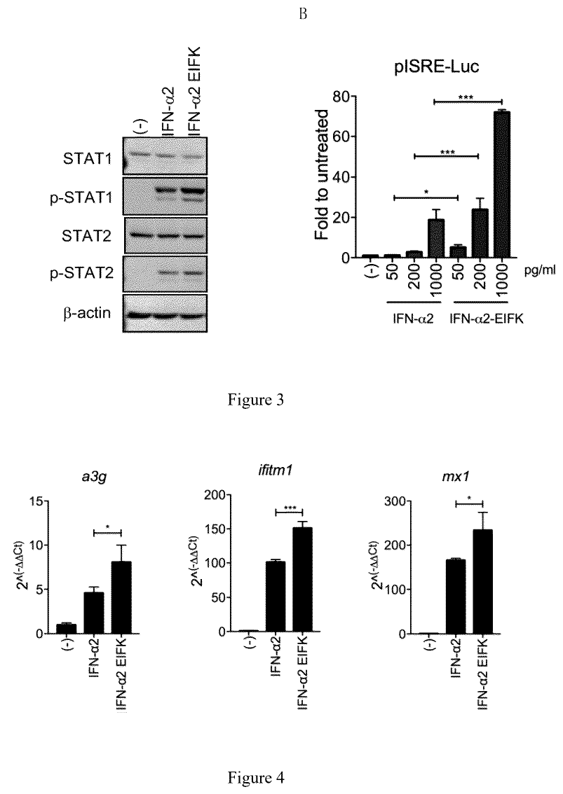

Purified human IFN-α2 and IFN-α2-EIFK recombinant proteins were used to treat HepG2-NTCP (FIG. 2A) or PHH (FIG. 2B) infected with hepatitis B virus particles. The production of hepatitis B e-antigen (HBeAg) and DNA was inhibited to varying degrees.
(1) Culture of HepG2-NTCP cells: Routine culture was performed using DMEM medium (Gibco, supplemented with 10% fetal bovine serum, 100 U/ml penicillin, and 100 μg/ml streptomycin and incubated at 37° C. in a humidified atmosphere with 5% CO2. For HBV infection experiments, the infection medium used was: routine culture medium supplemented with 2.5% DMSO. PHH cells were purchased from Shanghai Rild Biological Co., Ltd. and cultured in a specialized commercial medium.
(2) Hepatitis B virus used for infection was purified by the present laboratory. After collecting the HepAD38 supernatant, it was concentrated approximately 100-fold using a PEG8000 precipitation method, and infection was carried out at a concentration of 200 copies/cell.
(3) Three days after cell infection, human IFN-α2 or IFN-α2-EIFK mutant interferon was added to each group of cells respectively (interferon concentration in the HepG2-NTCP system was 0.2 or 1 ng/ml; interferon concentration in the PHH system was 0.04 or 0.2 ng/ml), and the medium was changed and the cells were re-treated every 72 hours of incubation.
(4) On day 9 post-infection, the supernatant of the cells was collected to detect the viral antigen marker HBeAg by ELISA, and viral DNA production in the supernatant was quantified by qPCR using specific HBV primers.
(5) In both HBV-infected cell models, the results demonstrated that IFN-α2-EIFK exhibited superior inhibition to those of IFN-α2 in inhibiting the production of HBV antigen and viral DNA (as shown in FIG. 2).
(6) Western blotting analysis further revealed that, compared to IFN-α2, IFN-α2-EIFK more potently activates the classical JAK-STAT1/STAT2 signaling pathway, i.e., stimulate the phosphorylation levels of STAT1 and STAT2 to a higher degree. Additionally, IFN-α2-EIFK induced stronger activation of the Interferon-Stimulated Response Element (ISRE) (as shown in FIG. 3).
(7) Further investigation of intracellular ISG induction after treating HepG2-NTCP cells with IFN-α2 and IFN-α2-EIFK for 6 hours showed that IFN-α2-EIFK can induce a higher magnitude of ISGs compared to IFN-α2 (as shown in FIG. 4). This increased ISG induction may be related to its better antiviral effect.

The above examples illustrate that the present invention involves the preparation of wild-type and mutant interferons through cloning and purification. The anti-HBV effects of human IFN-α2 and the IFN-α2-EIFK mutant were compared. The experimental results show that IFN-α2-EIFK, obtained by mutating amino acid residues related to IFNAR1 to change the affinity of IFN-α2 for IFNAR1, demonstrated a more significant anti-HBV effect in inhibiting HBV antigen and DNA levels. This enhanced effect is correlated with its ability to stimulate higher levels of STAT1 and STAT2 phosphorylation, activate higher levels of ISRE, and induce higher levels of antiviral-related ISGs. This method of mutating IFN-α interferon receptor binding-related sites will provide new approaches for developing new interferons for antiviral therapies. The development of novel interferons has good application and development prospects.

SEQUENCE LISTING

SEQ ID NO. 1
The amino acid sequence of IFN-α2
CDLPQTHSLGSRRTLMLLAQMRRISLFSCLKDRHDFGFPQEEFGNQFQK
AETIPVLHEMIQQIFNLFSTKDSSAAWDETLLDKFYTELYQQLNDLEAC
VIQGVGVTETPLMKEDSILAVRKYFQRITLYLKEKKYSPCAWEVVRAEI
MRSFSLSTNLQESLRSKE
SEQ ID NO. 2
The amino acid sequence of IFN-α2-EIFK
CDLPQTHSLGSRRTLMLLAQMRRISLFSCLKDRHDFGFPQEEFGNQFQK
AETIPVLHEMIQQIFNLFSTKDSSAAWDETLLEKFYIELFQQLNDLEAC
VIQGVGVTETPLMKEDSILAVKKYFQRITLYLKEKKYSPCAWEVVRAEI
MRSFSLSTNLQESLRSKE
SEQ ID NO. 3
The amino acid sequence of IFN-α14
CNLSQTHSLNNRRTLMLMAQMRRISPFSCLKDRHDFEFPQEEFDGNQFQ
KAQAISVLHEMMQQTFNLFSTKNSSAAWDETLLEKFYIELFQQMNDLEA
CVIQEVGVEETPLMNEDSILAVKKYFQRITLYLMEKKYSPCAWEVVRAE
IMRSLSFSTNLQKRLRRKD

1. A human interferon alpha receptor 2 (IFN-α2) mutant, comprising the amino acid sequence of SEQ ID NO: 1 with one or more substitutions selected from D82E, T86I, Y89F, and R120K.

2. A method for treating hepatitis B virus infection in a subject comprising administrating the human IFN-α2 mutant according to claim 1 to the subject.

3. A method for reducing the level of hepatitis B surface antigen (HBsAg), hepatitis B e-antigen (HBeAg), and/or viral genomic DNA of hepatitis B virus in a subject with hepatitis B virus infection, comprising administrating the human IFN-α2 mutant according to claim 1 to the subject.

4. A method for inhibiting the production of HBsAg, HBeAg, and/or viral DNA of hepatitis B virus in hepatocytes infected with hepatitis B virus, comprising administrating the human IFN-α2 mutant according to claim 1 to the hepatocytes.

5. The human IFN-α2 according to claim 1, wherein at the same working concentration, the anti-hepatitis B virus activity of the IFN-α2 mutant is higher than that of IFN-α2.

6. The method according to claim 4, comprising activating the JAK-STAT1/STAT2 pathway and the Interferon-Stimulated Response Element (ISRE), and inducing a high level of an antiviral molecule.

7. The human IFN-α2 mutant according to claim 1, wherein the IFN-α2 mutant comprises the substitutions of D82E, T86I, Y89F, and R120K.

8. The human IFN-α2 mutant according to claim 1, wherein the IFN-α2 mutant comprises the amino acid sequence set forth in SEQ ID NO: 2.

9. The human IFN-α2 mutant according to claim 1, wherein the effective concentration of the IFN-α2 mutant to achieve an antiviral effect is 10-fold less than that of wild-type IFN-α2.

10. The method according to claim 2, wherein the hepatitis B virus infection is chronic hepatitis B virus infection.

11. The method according to claim 6, wherein the antiviral molecule is Interferon-Stimulated Genes (ISGs).


SEQUENCE LISTING (3 sequences)

SEQ ID NO: 1 - protein, 165 aa, Homo sapiens [sequence omitted]

SEQ ID NO: 2 - protein, 165 aa, Artificial Sequence
  Chimeric sequence derived from human IFN-A14 and  IFN-A2
CDLPQTHSLGSRRTLMLLAQMRRISLFSCLKDRHDFGFPQEEFGNQFQKAETIPVLHEMIQQIFNLFSTKDSSAAWDETLLEKFYIELFQQLNDLEACVIQGVGVTETPLMKEDSILAVKKYFQRITLYLKEKKYSPCAWEVVRAEIMRSFSLSTNLQESLRSKE

SEQ ID NO: 3 - protein, 166 aa, Homo sapiens
CNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD